(19) United States
(12) Patent Application Publication
(10) Pub. No.: US 2009/0324549 A1
(43) Pub. Date: Dec. 31, 2009

(54) PROTEINS AND/OR PEPTIDES FOR THE PREVENTION AND/OR TREATMENT OF NEURODEGENERATIVE DISEASES

(75) Inventors: Giorgio Stefano Battaglia, Milano (IT); Denise Locatelli, Calusco D'Adda (Bergamo) (IT); Alfredo Martini, Milano (IT); Veronica Setola, Milano (IT)

Correspondence Address: AMSTER, ROTHSTEIN & EBENSTEIN LLP, 90 PARK AVENUE, NEW YORK, NY, 10016, US

(73) Assignee: FONDAZIONE I.R.C.C.S. ISTITUTO NEUROLOGICO "CARLO BESTA", MILANO (IT)

(21) Appl. No.: 12/524,322

(22) PCT Filed: Sep. 3, 2007
(86) PCT No.: PCT/EP2007/006660

§ 371 Date: Jul. 23, 2009

(30) Foreign Application Priority Data
Jan. 29, 2007 (IT) MI2007A000127

(51) Int. Cl. A61K 35/74; C07K 2/00; C07K 16/00; C07H 21/00; A61K 38/02; A61K 38/18; C12N 1/21; A01K 67/033; A01N 1/00; C12N 5/10; G01N 33/68

(52) U.S. Cl. 424/93.2; 530/300; 530/389.1; 530/388.1; 536/23.1; 514/2; 514/12; 435/252.3; 800/9; 435/1.1; 435/325; 436/86

(57) ABSTRACT

Proteins and/or peptides originate from the gene which results from the retention of the intron 3 of the gene SMN identified in the gene bank with the access number AY876898 with use for the diagnosis and/or prevention and/or treatment of neurodegenerative diseases.

The present invention refers to proteins and/or peptides for the study and/or diagnosis and/or prevention and/or treatment of neurodegenerative diseases.

In particular, the present invention reveals a new protein transcript and product of the SMN gene.

The overexpression of the new protein in vitro shows a marked functional effect on axonal growth, both on neuronal and non-neuronal phenotype cells.

This axonogenic effect opens interesting therapeutic prospects for neurodegenerative diseases, among others which can benefit from an induction of axonal growth.

Spinal muscular atrophy is an autosomal recessive disease characterised by a selective neuronal degeneration leading to respiratory and progressive amyotrophic paralysis (Pearn, 1980). It represents the most common genetic cause of childhood death, with an incidence of 1:6000/1:10000 live births and a carrier frequency of 1:35 individuals (Feldkotter et al., 2002). The gene responsible for SMA is SMN or survival motor neuron (Lefebvre et al, 1995). In humans, the SMN gene is present in two copies, the telomeric gene (or SMN1) and the centromeric gene (or SMN2) on the chromosome 5q13. The gene SMN1 is the disease gene of the SMA. There are in fact homozygote mutations or deletions of such gene in over 98% of the patients affected by SMA (Lefebvre et al, 1998). The gene SMN2 on the other hand modulates the severity of the disease: it has in fact been observed that in the SMA forms with lighter phenotype several copies of the SMN2 gene are present (Vitali et al, 1999). Both genes can produce an identical, functionally active protein, of 294aa, which does not have homologies with other known proteins (Coovert et al, 1997; Lefebvre et al, 1997). Nevertheless, the primary transcript of the gene SMN1 is the functionally-active full-length SMN2 protein (FL-SMN), while the gene SMN2, due to a C/T transition in the gene sequence which alters the splicing pattern (Gennarelli et al, 1995; Lefebvre et al, 1995; Lorson et al, 1998, 1999; Lorson & Adrophy, 2000), mainly produces a protein lacking exon 7 (Δ7-SMN), which is more instable and of lesser physiological importance.

Notwithstanding that the motor neurons are the specific target of this disease, the protein FL-SMN is expressed everywhere in the organism. It is localised in large multimeric complexes at the nuclear level (in structures called “gems” and in the coiled bodies) and in the cytoplasm of all cell types, comprising the motor neurons (Liu & Dreyfuss, 1996; Battaglia et al, 1997). The currently confirmed roles for the FL-SMN protein have it involved in fundamental functions for every cell type, such as the assembly of the snRNPs and the splicing of the pre-mRNA (Liu et al, 1997; Fischer et al, 1997; Pellizzoni et al, 1998; Pauskin et al, 2002; Yong et al, 2004; Carissimi et al, 2005; Grimmler et al, 2005) and the regulation of the gene transcription (Strasswimmer et al, 1999; Williams et al, 2000; Pellizzoni et al, 2001; Young et al, 2002). In recent years, the localisation of the SMN protein was demonstrated on the growth cone and axon level, along with the association of cytoskeletal elements in this zone (Pagliardini et al, 2000; Fan & Simard, 2002; Zhang et al, 2003; McWhorter et al, 2003; Rossoll et al, 2002, 2003; Sharma et al, 2005; Zhang et al, 2006; Carrel et al, 2006). The importance of the association between the protein SMN and cytoskeletal elements for SMA is moreover underlined by the presence of an accumulation of neurofilaments in the neuromuscular junctions, both in patients affected by SMA (Lippa & Smith, 1988) and in SMA transgenic mice (Cifuentes-Diaz et al., 2002).

It is still not clear why the reduction of the FL-SMN protein level involves the selective degeneration which distinguishes SMA. The protein FL-SMN could have specific functions on the motor neuron level, for example, interacting with proteins which are particularly expressed in this cell type, but these presumed functions or proteins have not been identified up to now. On the other hand, the possible presence of further protein isoforms of the SMN gene with a specific role at the motor neuron level could help us understand the pathogenic mechanisms underlying SMA.

The technical task of the present invention is essentially that of identifying new proteins and/or peptides for the study and/or diagnosis and/or prevention and/or treatment of neurodegenerative diseases, particularly in humans.

This technical task is attained by proteins and/or peptides in accordance with claim 1.

Other aspects of the present invention are shown in the subsequent claims.

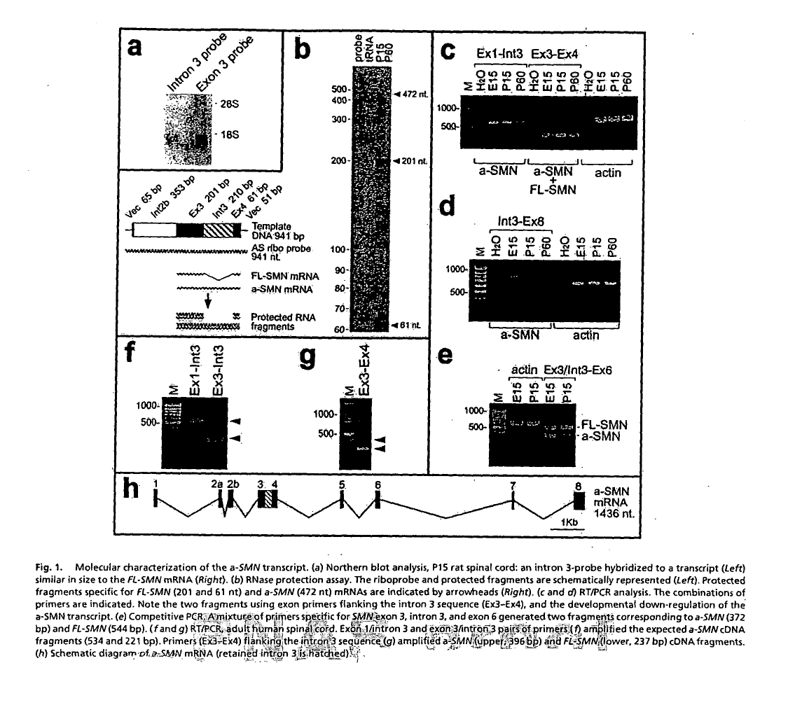

The invention is described by making reference to the attached FIGS. 1-6.

We have recently identified and characterised a new transcript of the gene SMN (GenBank accession No. AY876898) which originates from the retention of the intron 3 of the SMN gene and encodes for a protein which we have called axonal SMN (a-SMN), expressed in the neuronal and extra-neuronal tissues mainly in the early development stages. In rat spinal cord, a-SMN is selectively expressed in the motor neurons and is mainly localised in the axons. The selective axonal expression of a-SMN is confirmed by the overexpression of the protein in in vitro cell systems. These experiments have surprisingly demonstrated that the protein a-SMN is capable of stimulating axonogenesis in a time-dependent manner and that such effect can be shown both in neuronal and in non-neuronal phenotype cells, such as HeLa.

The N-terminal portion of the a-SMN protein is responsible for the axonal localisation, while the C-terminal portion is essential for the axonogenesis.

Given the specific localisation and axonal function of a-SMN, it is possible that it can carry out a role in human motor neurons. Moreover, since we have demonstrated that the human a-SMN is a specific product of the SMN1 gene, i.e. the SMA disease gene, its absence could be a crucial event in the comprehension of the SMA etiopathogenesis.

A new protein isoform was therefore discovered of the SMN gene, the disease gene of spinal muscular atrophy or SMA.

We obtained first suggestive data of the existence of this new protein isoform with a molecular biology technique called RACE-PCR; in fact, by using oligonucleotides partially overlapping on exons 1 and 3 of the gene SMN, we have isolated from the polyadenylated portion of rat spinal cord mRNA a transcript containing the entire sequence of the intron 3.

The transcript containing the intron 3 was called axonal-SMN or a-SMN.

Northern Blot, RNA-Protection or PCR experiments with oligonucleotides on the intron 3 and exon 1 or exon 8 (FIG. 1a-e) indicate that the new a-SMN messenger RNA is entirely composed of exons encoding for the FL-SMN protein and by an intron (intron 3) retained inside the sequence. RACE-PCR and RT-PCR experiments carried out on the polyadenylated human spinal cord portion and on the human myeloid cell line NB4 have not only confirmed the existence of the messenger a-SMN also in humans (FIG. 1f-g) but have also shown that the human mRNA of a-SMN is mainly expressed by the gene SMN.

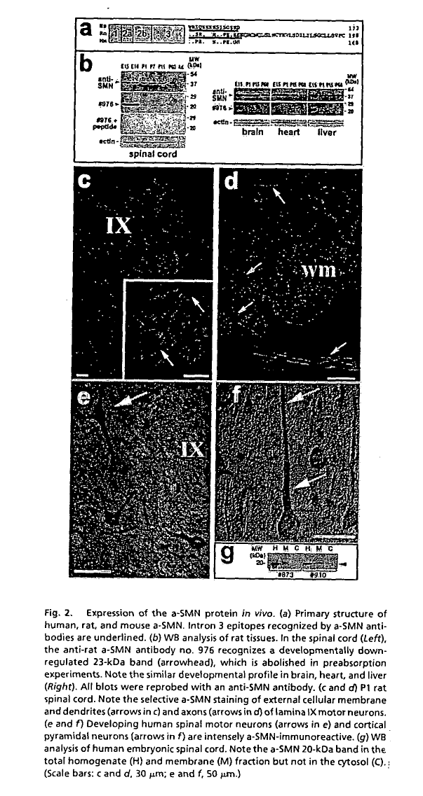

At this point, we verified the expression and subcellular localisation of the new in vivo a-SMN protein, since the presence of a new transcript does not necessarily demonstrate its effective translation into protein. Moreover, given the considerable homology between the protein a-SMN and the already known protein FL-SMN, we verified its biological role, and then its functional importance, by means of overexpression experiments in in vitro cell cultures. For the first point, we have produced antibodies specifically directed against the amino acid sequence of the a-SMN protein (FIG. 2a) encoded by human and rat intron 3, capable therefore of distinguishing the protein a-SMN from the protein FL-SMN both in Western Blot experiments and immunocytochemical experiments. These antibodies recognise, in Western Blot, protein bands of the expected molecular weight (about 23 kDa in rat and 20 kDa in man) in the rat spinal cord, brain, liver and heart (FIG. 2b), and in the human embryonic spinal cord (FIG. 2g), which are completely absorbed after the pre-incubation with the corresponding immunogenic peptide (FIG. 2b). Both in rat and man, the a-SMN protein is more greatly expressed during prenatal development, and then subsequently decreases its expression, as already verified for the a-SMN messenger. The confocal immunofluorescence and immunocytochemical experiments have shown that the a-SMA is selectively expressed by the spinal motor neurons in the rat and by the spinal motor neurons and cortical pyramidal neurons in humans (FIG. 2c-f). At the subcellular level, the motor neurons are characterised by an intense immunofluorescence at the axon level exiting from the spinal cord which forms the ventral roots, and this subcellular localisation detail justifies the name which we conferred to the protein (a-SMN, for axonal SMN).

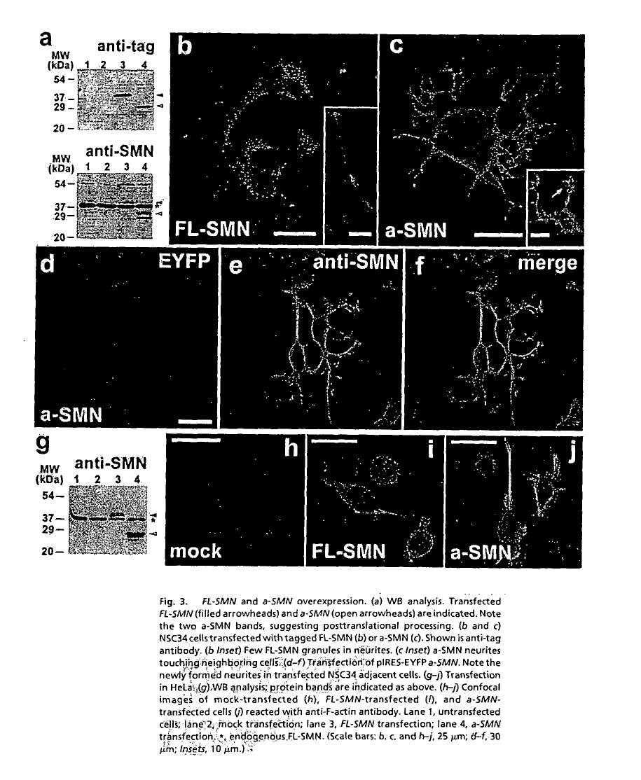

Regarding the second point, the functional role of a-SMN, we have carried out transfection experiments in cells NSC34 and HeLa and compared the effect of a-SMN with that of the already-known FL-SMN isoform on both cell type (FIG. 3a-f). The NSC34 are a motor neuronal line which maintains most of the characteristics of the primary motor neurons (Cashman et al. 1992; Simeoni et al. 2000), while the HeLa are an epithelial cell line of human uterine cervix without any neuronal characteristic. While the transfection with the FL-SMN protein does not cause any modification of the cell morphology, as already reported in literature for other cell systems (Pellizzoni et al., 1998; Cisterni et al 2001; Le et al, 2005), surprisingly the overexpression of a-SMN determines dramatic changes of the morphology of the cells NSC34. In fact, a-SMN is above all accumulated at the outer cell membrane level and induces the growth of an impressive number of long neuritic extensions which are radially developed in all directions from every single transfected cell.

These results were also confirmed in HeLa cells (FIG. 3g-j). The transfection with a-SMN, but not that with the empty vector nor with FL-SMN, also causes in HeLa cells the formation of cell processes similar to filopodia, relatively long and F-actin positive. These experiments in HeLa cells, phenotypically without neuritic extensions, demonstrate the dominating effect of a-SMN in determining the growth of cell extensions, with axonal growth cone characteristics given their positivity for F-actin, axonal growth cone marker (Fan & Simard 2002).

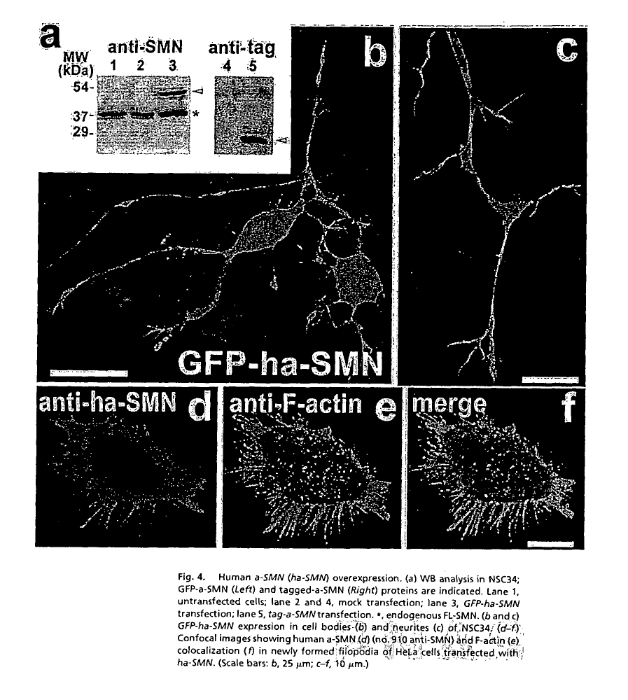

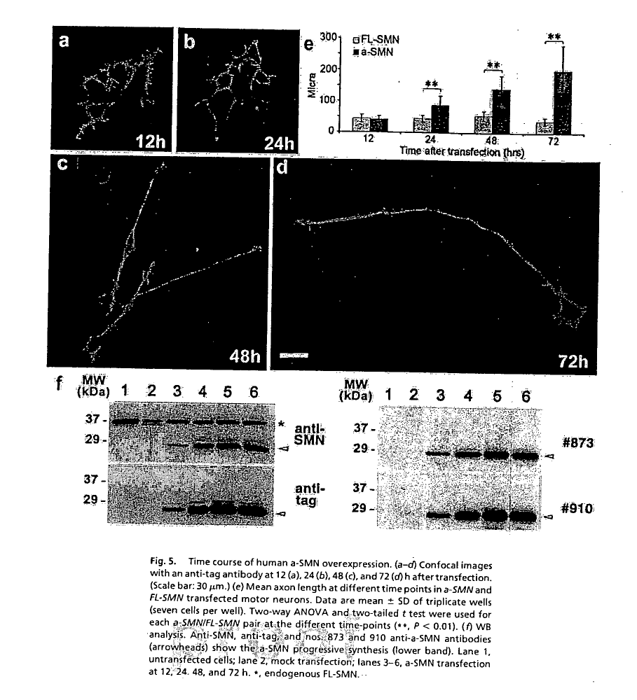

The functional characteristic of stimulus of axonal or axonal-like growth is possessed both by the rat and human a-SMN isoform (FIG. 4a-f). To verify the expression kinetics and the functional effect over time of the a-SMN human protein, we have carried out a transient transfection time course experiment. We have verified that the progressive expression of the human a-SMN protein accompanies progressive modifications of the motor neuronal cell morphology. Indeed, after 12 and 24 hours, the transfected motor neurons have a multipolar aspect with neurites which extend in all directions; after 48 hours the cells are mainly of bipolar morphology, the number of growing neurites has decreased, but their length is considerably increased; after 72 hours, finally, most of the transfected cells show a unipolar morphology, with the growth of particularly long axons (FIG. 5).

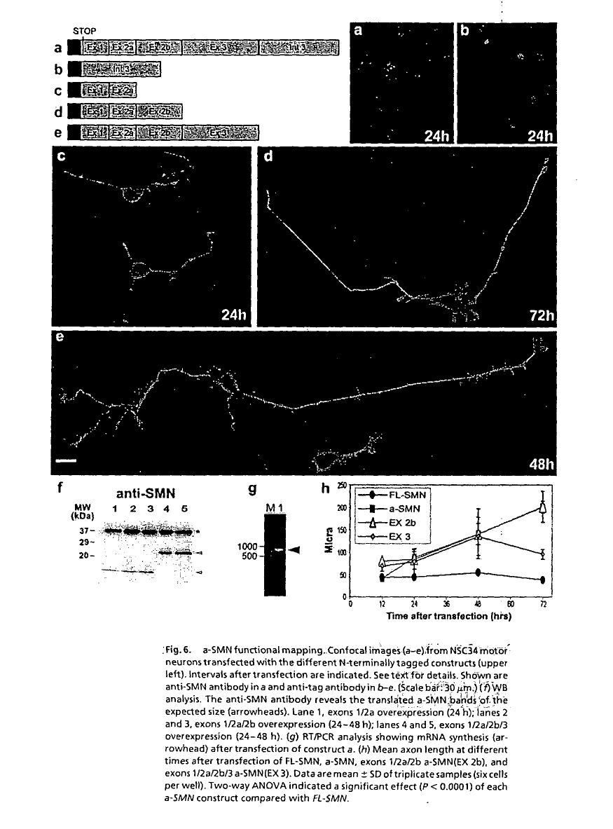

Finally, to explain the discrepancy between the a-SMN and FL-SMN structural resemblance (they differ only by a C-terminal part, but are identical in their N-terminal part), and their significantly different functional effect, we wished to identify the a-SMN epitope responsible for the induction of the axonogenesis. For this, we carried out a functional mapping of a-SMN by transfecting the NSC34 motor neurons with different constructs encoding for smaller parts of a-SMN (FIG. 6). The overexpression of a construct containing an in frame stop codon (which then leads to the synthesis of the mRNA but not of the related protein) does not induce any morphological change (FIG. 6a). Also, the overexpression of the peptide encoded by the intron 3 determines the accumulation of large intracytoplasmic granules without any modification of the cell morphology (FIG. 6b). The peptide encoded by the exons 1/2a is accumulated at the neuritic extension level, without however causing axonal growth modifications (FIG. 6c). On the other hand, the overexpression of the peptides encoded by the exons 1/2a/2b or 1/2a/2b/3 not only determines the accumulation of the corresponding proteins at the axon level but also the induction of a progressive axonal growth (FIG. 6d-e).

This data shows that: 1) the synthesis of the a-SMN protein is necessary for the axonal sprouting; ii) the N-terminal part of a-SMN, i.e. the sequence encoded by the exons 1-2a, is important for the axonal localisation; iii) the C-terminal portion of a-SMN, i.e. the sequence encoded by the exons 2b-3, is essential for axonogenesis; iv) finally, the retention of the intron 3 is only important for providing a stop codon necessary to produce a SMN polypeptide truncated at the ex3/ex4 junction, which acquires, due to this structural characteristic, axonogenesis stimulation properties.

It is not yet known which are the proteins which interact with a-SMN and which are the molecular mechanisms underlying its properties in the axonogenesis induction. a-SMN maintains, in its own peptide sequence, the interaction site with SIP1/Gemin2 (Liu et al, 1996). We have discovered that the cotransfection of a-SMN and SIP1/Gemin2 considerably increases the axonogenesis properties of a-SMN, indicating a functional interaction between these two proteins.

From that set forth above, it appears clear that our invention opens new prospects in the context of the therapeutic strategies of neurodegenerative diseases, particularly for that regarding the possibility of a gene therapy (Azzouz et al, 2004a-b).

A first application of our invention consists of the production, according to methods known to those skilled in the art, of vectors containing the entire a-SMN protein or parts thereof or derived peptides of the same and the use of said vectors for transfecting suitable cell lines.

In our embodiment, the preferred vectors contain the C-terminal portion of the a-SMN responsible for its axonogenic properties and the preferred cell line is represented NSC34 cells, engineered with a Tet/on or Tet/off system, such to permit the stable and conditioned transfection of a-SMN, but the choice of this cell line is non-limiting due to the adoption of other appropriate cell lines.

This first application permits the creation of a disease cell model, useful for the study of the molecular mechanisms responsible for the motor neuronal degeneration in the SMA and in other neurodegenerative diseases characterised by motor neuronal death.

A further application consists of the use of the construct/constructs in accordance with the preceding application, according to methods known by those skilled in the art, for transfecting bacterial cells for the production and purification of polyclonal and/or monoclonal antibodies. Such antibodies can be used in the diagnosis of SMA, and in other pathologies of humans and/or animals, characterised by motor neuronal degeneration. Such antibodies can also be used for the in vitro and in vivo detection of axonal and dendritic extensions.

A further application consists of the use of the construct in accordance with the preceding applications as vector for the production, according to methods known to those skilled in the art, of transgenic non-human mammals capable of expressing increased quantities of a-SMN protein, and on the other hand transgenic non-human mammal characterised by a downregulation of the a-SMN protein.

In our embodiment, the preferred animal is the mouse, but the animal could also be chosen from among other appropriate animal species (including non-mammals). This application permits the creation of animal disease models, useful for the study of the pathogenesis of the motor neuronal degeneration in the SMA and in other neurodegenerative diseases characterised by motor neuronal death.

A further application of our invention consists of the production, according to methods known by those skilled in the art, of vectors containing the entire a-SMN protein or parts thereof or derived peptides of the same, and the SIP1/Gemin2 protein and the use of said vectors for transfecting the cell lines mentioned for the preceding applications. This application permits the creation of a further disease cell model, in which it is possible to study the molecular mechanisms underlying the axonogenic properties of the a-SMN protein.

A further application of our invention consists of the use of the vectors according to the preceding applications for the generation, according to methods known by those skilled in the art, of viral vectors to be used in gene therapy, with replacement or overexpression of the a-SMN protein, of: i) the cell models of the SMA/neurodegenerative diseases described above; ii) the transgenic animal models of the SMA or other neurodegenerative diseases characterised by motor neuronal degeneration; iii) other transgenic animal models of the SMA or other neurodegenerative diseases characterised by motor neuronal degeneration; iv) patients affected by various clinical SMA phenotypes; v) patents affected by other neurodegenerative diseases characterised by motor neuronal death; vi) patients affected by clinical conditions characterised by motor neuronal death or by selective loss of the motor axonal pathways with consequent peripheral paralysis or trauma.

The above-described applications are reported as example and are not in any manner limiting of the developments of our invention.

For example, the proteins and/or peptides obtained by means of synthesis or genetic engineering techniques in accordance with the present invention can be conjugated with peptide sequences with intracellular carrier function, such as the TAT peptide (but not only), with use for the prevention and treatment of neurodegenerative diseases in humans and in experimental animals.

### EXAMPLES

### Example 1

### Identification of a new protein isoform of the gene SMN, a-SMN

We initially conducted RACE-PCR experiments on the polyadenylated portion of mRNA of rat spinal cord. Using oligonucleotides partially overlapping on the exon 1 and 3 of the gene SMN, we isolated a transcript containing the entire sequence of the intron 3, in addition to cDNAs corresponding to the FL-SMN form. The transcript containing the intron 3 was called axonal-SMN or a-SMN.

In detail, in order to obtain rat spinal cords, male Sprague-Dawley rats were decapitated, after anaesthesia with diethyl ether, on the 15^{th} and 60^{th} day of post-natal life (P15 and P60). For the embryonic tissues, pregnant rats were anesthetised with chloral hydrate on the fifteenth day of gestation (E15). The embryos were quickly drawn from the uterus and immersed in an oxygenated medium for dissection under surgical microscope. The drawn spinal cords were immediately frozen by immersion in liquid nitrogen or on dry ice and preserved at −80° C. until use. For the extraction of the total RNA, the spinal cords were homogenised in 10 volumes of 4.4M guanidine isothiocyanate and 0.7% β-mercaptoethanol, and centrifuged for ten minutes at 10,000 g at 18° C. The supernatant was passed through several times with a 20 G syringe and loaded on CsCl phase for overnight centrifugation with balancing rotor at 100,000 g, at 18° C. The resulting aqueous phase was newly precipitated with 3M Na-acetate, pH 5.2, and 2 volumes of 100% EtOH in dry ice and centrifuged for 5 minutes at 10,000. The resulting pellet was washed in 70% EtOH, centrifuged for 2 minutes at 10,000 g and left to evaporate in air or in speed-Vac (Concentrator 5301, Eppendorf), for the resuspension in H₂O and subsequent spectrophotometer (Gene Quant, Amersham).

The portion corresponding to polyA⁺mRNA was obtained by purifying the total RNA with Dynabeads Oligo(dT)₂₅(DYNAL). The Oligo dTs are covalently bound to the surface of the Dynabeads, magnetic spheres of very small diameter. The total RNA was appropriately diluted, mixed 1:1 with a bond buffer (20 mM pH 7.5 Tris-HCl, 1M LiCl, 2 mM EDTA) and heated to 65° C. for 2 minutes to permit the denaturing. Subsequently the mixture was cooled on ice and mixed with Dynabeads Oligo(dT)₂₅, appropriately washed by the preserving solution, stirring for 5 minutes at room temperature. Due to the aid of a magnetic support, the pellet was recovered which was formed by magnets beads and polyA⁺mRNA. The pellet was resuspended in washing buffer (10 mM Tris-HCl, pH 7.5, 0.15M LiCl, 1 mM EDTA) and recovered by newly putting the test tubes containing the samples in the magnetic support. The supernatant, containing highly purified polyA⁺mRNA, was drawn, spectrophotometrically metered and preserved at −80° C. until use in the subsequent analyses.

The actual presence of this new transcript (i.e. a messenger RNA capable of producing a protein) was subsequently demonstrated by means of Northern Blot, RNase protection assay and RT-PCR experiments, conducted on the polyadenylated portion of RNA (containing therefore only messenger RNA) extracted from rat and human spinal cord. For the Northern Blot Analysis, 10 μg of polyA⁺mRNA of rat spinal rat was loaded on a gel with 1.5% agarose-formaldehyde and transferred on membrane (Gene Screen nylon, NEN). The hybridisation was carried out on probes radiomarked with α³²P-dCTP obtained by means of PCR on the exon 3 (for the recognition of the FL-SMN and a-SMN messenger) or on the intron 3 (for a-SMN). The specific cDNA probe for the intron 3 confirmed the presence of a transcript different from the known FL-SMN transcript, but similar thereto by molecular weight (FIG. 1a).

These results were confirmed by RNase protection experiments: a probe radiomarked with antisense RNA corresponding to the genomic sequence of the intron 2b, exon 3, intron 3 and exon 4 protected (i.e. blocked the degradation due to RNase) three fragments, of which two derive from the exons 3 and 4 of the messenger for FL-SMN, and the third from the exon 3/intron 3/exon 4 sequence of the a-SMN messenger. These experiments demonstrate that the new messenger of a-SMN originates from the same genomic strand of the messenger for FL-SMN, and that it is moreover down-regulated during its development (FIG. 1b).

In detail, the genomic DNA was extracted from P15 rat thymus. A PCR fragment which extended from the Int2bS (5′-aacctgatggactagaggatccccct-3′)-Ex4AS (5′-ctttacttctgagcgatctggaggag-3′) was cloned in the vector pBluescript II KS for the in vitro transcription T3/T7 and the marking with α³²P-dCTP. 5 μg of polyA⁺mRNA of P15 and P60 rat spinal cord or tRNA, as negative control, were protected with the hybridisation of sense and antisense radiomarked probes before being digested with RNase A/T according to the instructions of the kit RPAIII™ (Ambion). RNA century markers (Ambion) were marked with α³²P-dCTP and used for the identification of the molecular weights.

RT-PCR experiments with oligonucleotides on the intron 3 and exon 1 or exon 8 (FIG. 1c-d) indicate that the new transcript a-SMN is composed of all exons encoding for the protein FL-SMN and by an intron (intron 3) retained inside the sequence. The presence of the intron 3 in the gene sequence of a-SMN was confirmed by the fact that: i) oligonucleotides on the exon 3 and exon 4 amplify two different DNA fragments corresponding to the mRNA of a-SMN, which retains the intron 3, and to the mRNA of FL-SMN (FIG. 1c); ii) competitive PCR experiments with oligonucleotides on the exon 3 and intron 3/exon 6 indicate the existence of the transcripts for FL-SMN and a-SMN. Overall, the experiments conducted on the rat demonstrate that the messenger for a-SMN (schematically represented in FIG. 1h) constitutes only a small portion of the total pool of messengers of the SMN gene, and that it is clearly down-regulated during the development from E15 to P15 if compared with the transcript for FL-SMN (FIG. 1e).

RACE-PCR and RT-PCR experiments were conducted on the polyadenylated portion of human spinal cord and on the human myeloid cell line NB4 (Pisano et al, 2002). The RT-PCR experiments have shown the existence of the a-SMN transcript even in humans (FIG. 1f-g). The experiments of 3′-RACE-PCR with oligonucleotides on the intron 3 show that the human mRNA of a-SMN is mainly expressed by the gene SMN1. In fact, the sequence analysis of over 60 clones, obtained by the mRNA of NB4 cells, show that all clones derive from the gene SMN1, given that a C was present in position +6 of the exon 7 and a G present in position +233 in the exon 8. About two-thirds of the clones examined showed skipping of the SMN gene exon 5. The fact that, in the same experimental conditions, oligonucleotides at the exons 2b and 3 level amplified fragments corresponding to the messenger for FL-SMN and Δ7-SMN also from the SMN2 gene confirms the fact that the a-SMN transcript is mainly expressed by the SMN1 gene.

In detail, the polyA⁺mRNA portion of rat and human (commercially available from the BD Clontech company) was reverse transcripted with the following protocol: 1 μg polyA⁺mRNA, 1 μl Random primers (50 μM), 1 μl dNTPs Mix (10 mM each), sterilised water to reach a final volume of 12 μl. The mixture thus obtained was heated to 65° C. for 5 minutes and then cooled on ice. Subsequently the following were added: 4 μl 5× first strand buffer (250 mM Tris-HCl, pH 8.3, 375 mM KCl, 15 mM MgCl₂), 2 μl DTT (0.1M) and 1 μl of sterilised water. The solution was incubated at 25° C. for 2 minutes in SuperScript™ II RT (Invitrogen) and left to incubate for 50 minutes at 42° C. At the end of the incubation, the enzyme was inactivated by heating the reaction mixture for 15 minutes at 70° C. The total cDNA thus obtained was subjected to PCR experiments in order to amplify the desired fragment, making use of specific oligonucleotides on the rat gene sequence (Acc. No. U75369) and human gene sequence (Acc. No. NW_{—}047617) of the SMN gene: Ex1SI (5′-tgagcaggaagacaccgtgctgttcc-3′, nt 71-96), Ex3S (5′-tatctgatctgctttccccgacctgt-3′); Ex4AS (5′-ctttcctggtcctaatcccg-3′), Int3S (5′-tctggtgcactaaggtgttgagtgac-3′, nt 5558400-5558425), Ex8AS (5′-acagtttggctgacttccatgca-3′, complementary to nt 1118-1140). For the amplification reaction the following were used: 5 μl of cDNA (a quarter of the reverse transcription reaction) or plasmid DNA (for the amplification of the fragments to be cloned), 1 μl sense primer (10 μM), 1 μl antisense primer (10 μM), 0.5 μl Taq DNA polymerase (5 U/μl BD Advantage 2Polymerase mix), 0.5 μl dNTPs mix (10 mM each), 2.5 μl 10× PCR buffer (200 mM Tris-HCl, pH 8.4, 500 mM KCl) and sterilised water up to a final volume of 25 μl. The reaction mixture was subjected to an initial denaturing for 1 minute at 95° C., subsequently 33 cycles of 2 steps were executed: 1) denaturing for 30 seconds at 95° C.; 2) denaturing and coupling of the oligonucleotides for 1 minute at 68° C.; then an extension cycle for 10 minutes at 72° C. This last passage is fundamental to permit the cloning of the fragment obtained by means of a T/A cloning system inside appropriate vectors. The samples thus obtained were loaded on 1% agarose gel in TBE 1× (89 mM Tris, 89 mM boric acid, 2 mM EDTA, pH 8.3; Bio-Rad), with EtBr at 0.0001%, for a verification of the amplification accuracy of the desired fragments. The electrophoretic run was carried out at 80V in a solution of TBE 1×. The ethidium bromide, a mutagenic agent capable of inserting itself between the nitrogen bases of the DNA, permits the formation of a complex visible under UV, whose image was acquired with the instrument Fluor-S-Max MultiImager (Bio-Rad). The amplified fragments were purified on 1% ultrapure agarose gel (Invitrogen) and the corresponding bands were excised from the gel in order to proceed to the elution of the amplified portion by means of columns.

### Example 2

### Verification of the Expression and Subcellular Localisation of the New Protein Isoform of the Gene SMN, a-SMN

Since the presence of a new transcript does not necessarily demonstrate its biological importance, we deemed it necessary on the one hand to verify the expression and subcellular localisation of the new in vivo protein, and on the other study its biological role, over-expressing such protein in in vitro cellular cultures. This approach permits having the first relevant information on the function of the new protein and on its cellular and subcellular localisation, an important first step for understanding the role of SMA in pathogenesis.

To analyse the expression and the subcellular localisation of the new SMN protein in vivo (FIG. 2a), specific polyclonal antibodies were produced, directed against the amino acid sequence of the protein encoded by the human and rat intron 3.

In detail, the antibodies Nos. 937 and 976 directed against the intron carboxyl-terminal portion of rat a-SMN, and the antibodies Nos. 910 and 873, directed against the intron carboxyl-terminal portion of human a-SMN, were produced by the company Neosystem. The antigenic peptide was coupled with glutaraldehyde to the ovalbumin (the immunogenic carrier) by means of a tyrosine residue. The antigen/immunogenic carrier complex was injected in rabbits with monthly injection, before sacrificing the animals.

These antibodies specifically recognise the new isoform (and not FL-SMN) and are therefore capable of distinguishing the a-SMN protein from the FL-SMN protein in the cell. In Western Blot (WB) analysis, the antibodies directed against the rat sequence recognise a specific band at 23 kDa in the membrane portion of spinal cord, brain, liver and heart which is completely absorbed following incubation with the corresponding immunogenic peptide (FIG. 2b). Moreover, it can be observed that the protein expression is regulated during development, and in particular is more expressed during the motor neuronal development period (E15), with expression decreasing immediately after birth (P1) until its disappearance in the adult rat (P60).

In WB experiments conducted on human embryonic spinal cord, the two antibodies directed against the human a-SMN sequence recognise a specific band of the apparent molecular weight of about 20 KDa in the portions of the homogenate (H) and membranes (M) but not in the cytosol (C), it too completely absorbed if the antibody is preincubated with the corresponding immunogenic peptide (FIG. 2g).

Specifically, in order to obtain the rat tissues, male Sprague-Dawley rats were decapitated after anaesthesia with diethyl ether on the first, fifteenth and sixtieth day of post-natal life (P1, P15, P60). For the embryonic tissues, pregnant rats were anesthetised with chloral hydrate on the fifteenth day of gestation (E15). The embryos were rapidly drawn from the uterus, and inserted in an oxygenated medium for dissection under surgical microscope (Leitz). The drawn tissues were immediately frozen by immersion in liquid nitrogen or dry ice and preserved at −80° C. until use.

The human embryo spinal cords (15 gestation weeks) were obtained within an experimental project for the product of human fetal embryonic stem cells, approved by the Institute's Ethics Committee (Comitato Etico).

For the subcellular portioning, the tissues were homogenised in a Teflon-glass potter at 700 rpm in a buffer (4 ml/g of tissue) containing 1 mM DTT, 1 mM EGTA, 0.1 mM PMSF, 20 mM HEPES (pH 7.4) in the presence of a protease inhibitor cocktail (Complete™, Boehringer-Mannheim).

A part of the homogenate was centrifuged at 1,000 g for ten minutes. The precipitate, composed of linear cells, fragments of meninges and vessels, intact nuclei and other residues of the extraction operation, was eliminated while the supernatant was further centrifuged at 100,000 g for 45 minutes. The resulting pellet contains the cellular membranes and the intracellular organelles (M), while the supernatant corresponds to the cytosol (C) or soluble portion.

The protein extracts were subjected to discontinuous SDS-PAGE electrophoresis, according to the Laemmli method (1970) with appropriate modifications. Two different gel concentrations were used: 1) stacking gel (gel portion which permits the packing of the protein), composed of 2.9% acrylamide, 0.08% bis-acrylamide, 0.1% SDS, 0.1% TEMED, 0.05% ammonium-persulphate (AP) and Tris-HCl, pH 6.8; 2) running gel (gel portion which permits the separation of the proteins in relation to their apparent molecular weight), composed of 12% acrylamide, 3.2% bis-acrylamide, 0.1% SDS, 0.05% TEMED, 0.05% AP and 0.39% Tris-HCl, pH 8.8. The electrophoretic run was conducted at constant voltage: 50V in the stacking gel and 100V in the running gel. The running buffer used is composed of 0.2M glycine, 3.5M SDS, 0.028M Tris-HCl at pH 8.3-8.6. After the electrophoretic run, the proteins separated in the gel were transferred onto nitrocellulose membrane with constant amperage (180 mA) for one hour in a buffer composed of 0.192M glycine, 0.025M Tris, pH 8.3, and 20% methanol. To detect the transferred proteins, the membrane was coloured with Ponceau red (2% Ponceau, 30% Trichloroacetic acid), a reversible colorant of the proteins. Afterward the membranes were blocked with 10% skim milk in TBS (Tris buffered saline) at 4° C. and overnight, to prevent non-specific bonds, and were subsequently incubated for 90 minutes with the primary antibodies in 3% skim milk in TBS. After repeated washings in TBS-tween 20 (TBS-T) for 30 minutes, the nitrocellulose membranes were incubated with the secondary antibody conjugated with HRP (Horseradish peroxide) for 45 minutes (GAM, anti-mouse IgG, of the company Kierkegaard and Perry Labs diluted 1:10000 for the monoclonals and GAR, anti-rabbit IgG, of the company Sigma diluted 1:5000 in 3% skim milk for the polyclonal antibodies). After repeated washings with TBS-T for 40 minutes, the antigen-antibody complex was detected by using a chemiluminescence kit (ECL™, Amersham), by means of the use of sensitive emulsified strips, with variable time exposures according to the used antibody.

The confocal immunofluorescence experiments revealed an intense and selective marking of the motor neurons at the level of the lamina 1× of the anterior horns of the spinal cord (FIG. 2c-d). The marked motor neurons are characterised by an intense immunofluorescence at the level of the perinuclear regions, of the outer membrane level, and in particular of the dendritic and axonal processes in the portion near the cell body. Also at the level of the posterior horns, it is possible to appreciate an intense marking of the sensitive fibres afferent to the spinal cord. Moreover, at the white matter levels, fibres marked exiting from the spinal cord, constituting the dorsal roots, were quite evident (FIG. 2c-d).

Using the specific antibodies against ha-SMN on sections of human spinal cord and cortex (FIG. 2e-f), an intense and specific immunoreactivity can be observed of the pyramidal cortical neurons and motor neurons at the level of the external cellular membrane, and in a particular manner at the axonal level.

In detail, the rats, in the day after birth (P1), were first anesthetised with a solution of 4% chloral hydrate (1 ml/100 g of body weight), then intracardiacally perfused with a solution of 4% paraformaldehyde in PB. The cords were drawn, post-fixed in 4% paraformaldehyde for about 24 hours, then cut at the vibratome in coronal sections of 50 μm thickness. Such sections were collected in serial order and preserved in a solution of PB and 1% NaN₃.

For the experiments in immunofluorescence, sections of rat spinal cord P1 were pre-treated with 4% saccharose for 30 minutes then with 100% methanol for 30 minutes and frozen at −20° C. to facilitate the penetration of the antibody. The sections were incubated with 10% NGS serum (Normal Goat Serum) in PBS for 1 hour, in order to saturate the non-specific absorption sites, and incubated with the polyclonal anti-a-SMN primary antibody (No. 937, diluted 1:1000) and 1% NGS in 1× PBS, at 4° C. overnight. The sections are then incubated with the secondary fluorescent antibody Alexa Fluor® 546 GAR (Molecular Probes, Eugene, Oreg., USA; diluted 1:2000) for one hour and washed for three times (10 minutes for each washing) to eliminate the secondary antibody excess. Once the washings have been completed, the sections are mounted in water on slides, covered with Fluosave (Calbiochem, Darmstad; Germany) and examined with Bio-Rad Radiance 2100 confocal microscope. The images were subsequently processed with the Adobe Photoshop 7.0 program. The slides were preserved in the dark at 4° C. in order to minimise the decay of the fluorescent signal.

The human brain tissues were instead obtained following surgical removal from a patient not-affected by SMA, fixed by immersion in a solution of 4% paraformaldehyde at 4° C. for about 24 hours and cut at the vibratome in 50 μm thickness sections. The sections cut at the vibratome were protected in a 4% saccharose solution for 30 minutes at room temperature. After washing with phosphate buffer saline (PBS), they were pretreated with 1% H₂O₂ in PBS for 20 minutes, in order to neutralise the activity of the endogenous peroxidases.

The spinal cord included in paraffin was obtained by the Neuropathology Unit (Unita' di Neuropathologia) of the Istituto Neurologico “Carlo Besta” and cut at the microtome (Leica) in 5 μm coronal sections. The paraffin sections are deparaffined, hydrated, and then treated with boiling in 10 mM Sodium Citrate buffer, pH 6, for 5 minutes, using a microwave at 650-700W to recover the antigen immunoreactivity.

All sections were then washed again in PBS and incubated with 10% Normal Goat Serum (NGS) in PBS for 60 minutes to conceal the non-specific absorption sites. To this solution 0.2% Triton X-100 was added, a permeabilising surface-acting agent used to improve the penetration of the antibody. The sections were then incubated overnight at 4° C. with the anti ha SMN antibody (No. 910, diluted 1:1000) in a solution of 1% NGS in PBS. After 3 10-minute washings in PBS, the sections were incubated for 1 hour at room temperature with anti-rabbit biotinylated secondary antibody IgG diluted 1:200 in PBS (Vector Laboratories, Burlingname, Calif.), again washed in PBS for 30 minutes and incubated with the avidin-biotin complex conjugated with peroxidase (ABC, Vector Laboratories) or Extravidin (Sigma-Aldrich, St. Louis, Mo.) diluted 1:100 in PBS.

The colouration was obtained by incubating the sections in 0.075% DAB (3-3′-diaminobenzidine) and 0.002% H₂O₂ in 50 mM Tris HCl buffer. The subsequent analysis was carried out with optical microscope Nikon Microphot-FXA.

### Example 3

### Functional Significance of the New Protein Isoform of the Gene SMN, a-SMN

To evaluate the functional significance of the a-SMN protein, and compare it with the role undertaken by the isoform FL, we carried out transfection experiments in NSC34 cells (Neuroblastoma Spinal Cord). These cells belong to a hybrid motor neuronal line obtained by the fusion of a murine neuroblastoma line (N18TG2) with primary cultures of spinal motor neurons, coming from the spinal cord of mice embryos in the 12^{th}-14^{th} day of gestation (Cashman et al. 1992). Such cells show a neuronal phenotype, maintaining most of the characteristics of the primary motor neurons.

In detail, the NSC34 cells were maintained in the D-MEM medium (Dulbecco's Modified Eagle's Medium) added at the time of use with 5% FBS (Fetal Bovine Serum, Hyclone), 1 mM glutamine and antibiotics (potassium salt of penicillin G, Squibb, 100 UI/ml and streptomycin sulphate, Squibb, 100 μg/ml) and grown at 37° C. in a conditioned atmosphere (5% CO₂, 95% air) in 25 cm² flasks (Corning, Cambridge, Mass.) containing 7 ml of medium, periodically substituted every two or three days. Every week, the cells were mechanically removed in culture medium and replaced in new flasks, so to maintain a density of 5×10⁴ cells/flask.

The first step was the cloning of the cDNA of a-SMN and FL-SMN in the expression vector pcDNA4/HisMaxTOPO (T/A cloning®, Invitrogen) which permits the synthesis of a fusion protein (tag-FL-SMN and tag-a-SMN) composed of the cloned protein and by a tag sequence in terminal-amino position. This initial sequence, which is situated downstream of the first ATG codon, i.e. of the site where transcription begins, is recognised by specific antibodies (anti-tag), in this manner permitting the recognition of the exogenous protein after the transfection in in vitro systems. The cellular localisation and the biological activity of the a-SMN protein were compared with that of the isoform FL with WB techniques as well as confocal immunofluorescence and morphological analysis. The protein extracts of cells NSC34, respectively transfected with the isoform tag-FL-SMN and tag-a-SMN, were used for Western Blot experiments. The protein extracts of untransfected cells do not show any immunoreactivity to the anti-tag antibody since it recognises only the transfected protein. The anti-SMN antibody recognises in the untransfected cells a band with apparent molecular weight of 38 kDa, corresponding to the endogenous protein FL-SMN (FIG. 3a).

There is an analogous situation in cell extracts of cells transfected with the empty vector: there is no immunoreactivity for the anti-tag antibody, while the anti-SMN antibody recognises a band corresponding to the endogenous protein FL-SMN. Analysing the immunoreactivity of the protein extracts of NSC34 transfected with tag-FL-SMN, it is observed that the anti-tag antibody recognises a single band with apparent molecular weight of 41 kDa which corresponds to the fusion protein FL-SMN. The anti-body anti-SMN recognises two bands, with apparent molecular weight of 41 kDa and 38 kDa, which respectively correspond to the exogenous protein and to the endogenous protein (the contribution of the tag in the fusion protein is about 3 kDa more with respect to the endogenous protein) (FIG. 3a). In the protein extracts transfected with tag-a-SMN, the anti-tag antibody recognises two bands, both corresponding to the exogenous protein, with apparent molecular weight of 29 kDa and 27 kDa, respectively. Finally, the anti-SMN antibody recognises three immunoreactive bands: the first corresponds to the endogenous protein FL-SMN, with apparent molecular weight of 38 kDa, the successive both correspond to the protein tag-a-SMN, with apparent molecular weight of 29 kDa and 27 kDa, respectively. The presence of two bands with a small molecular weight difference, both corresponding to the protein tag-a-SMN, leads to the belief that posttranslational events occur in the carboxyl-terminal portion of the protein (FIG. 3a).

The confocal immunofluorescence experiments permit completing a morphological analysis, observing the biological effect generated by the transfection of in vitro tag-FL-SMN and tag-a-SMN. Both transfected proteins were detected with the anti-tag and anti-SMN antibodies. The cells NSC34 transfected with the full length protein show an evident accumulation of the protein itself on the large granule level, present both on the cytoplasmic and nuclear level, and a marking of lesser intensity on the neuritic level, but no modification is observed of the cell morphology (FIG. 3b). These results are in accordance with those reported for other cell systems (Pellizzoni et al 1998; Cisterni et al 2001; Le et al, 2005) after transfection of the protein FL-SMN. On the other hand, the transfection of tag-a-SMN causes evident changes in the morphology of the cells NSC34. The overexpressed protein tag-a-SMN is mainly accumulated on the cell membrane level and induces, in all transfected cells, the growth of a high number of particularly long neuritic extensions (100-200 microns), which are radially developed by the cell membrane itself and are strongly immunoreactive (FIG. 3c).

Since the NSC34 cells are motor neurons, they are induced to the differentiation towards a motor neuronal phenotype and thus to the emission of neuritic extensions, if subjected to certain stimuli, such as for example hydroxyurea (Simeoni et al 2000). To evaluate if the biological effect, observed by the transfection of a-SMN, is only that of accelerating this forming process, we used the cell line HeLa, of epithelial derivation, which does not have neuronal characteristics. It has a rounded form and lacks extensions on the cell surface.

The HeLa cells consist of an immortalised cell line and were obtained by epithelial carcinoma cells of the human uterine cervix transformed with human papilloma virus 18 (HPV 18). Also the HeLa cells were maintained in the medium D-MEM (Dulbecco's Modified Eagle's Medium) added at the time of use with 10% FBS (Fetal Bovine Serum, Hyclone), 1 mM glutamine and antibiotics (100 UI/ml potassium salt of penicillin G, Squibb, and 100 μg/ml sulphate streptomycin, Squibb) and grown at 37° C. in conditioned atmosphere (5% CO₂, 95% air) in Petri dishes (Corning, 100 mm×20 mm) containing 10 ml of medium. Every two or three days the HeLa cells, after having undergone a brief washing with 1× PBS (0.01M phosphate buffer, 0.0027M KCl, 0.137M NaCl, pH 7.4, preserved at 4° C.), were removed with Tripsin-EDTA (Gibco-Invitrogen) and newly placed in Petri dishes (100 mm×20 mm) so to ensure the maintenance of a cell density equal to about 7×10⁵ cells/plate.

The protein extracts of the HeLa cells, obtained after the transfection with tag-FL-SMN and tag-a-SMN, were used for Western Blot experiments and were detected with anti-SMN antibody (FIG. 3g). Analogous to that observed for the NSC34 cells, the anti-SMN antibody detects a single band corresponding to endogenous FL-SMN at the untransfected cells, or cells transfected with empty vector. The cell extracts of HeLa transfected with tag-FL-SMN show two immunoreactive bands which correspond, respectively, to the exogenous FL protein, with apparent molecular weight of 41 kDa, and to the endogenous form, with apparent molecular weight of 38 kDa (FIG. 3g). Analysing the immunoreactivity of the protein extracts of HeLa cells transfected with tag-a-SMN, it is possible to observe the presence of a band corresponding to the endogenous FL-SMN protein, with apparent molecular weight of 38 kDa, and two bands corresponding to the protein tag-a-SMN, with apparent molecular weight of 29 kDa and 27 kDa, respectively. Also in this case, the difference of molecular weight between the two bands corresponding to the protein a-SMN is probably due to a posttranslational processing at the carboxyl-terminal of the protein (FIG. 3g).

In order to obtain the protein extracts, the cell pellets NSC34 and HeLa were resuspended in the lisi buffer (0.1M sodium phosphate buffer, 0.2% Triton X-100, 0.1 mM EDTA, 0.2 mM PMSF, 1 μg/ml leupeptine, 1 μg/ml aprotinine) and subjected to 3 hot/cold cycles, each of which foresees 5 minutes in pulverised dry ice and 5 minutes at 37° C. in the thermostatic bath. The cell lysates were then centrifuged at 10,000 g for 5 minutes and the supernatant was recovered containing the desired protein extracts. The protein concentration was spectrophotometrically determined at the wavelength of 595 nm after the addition of the colorant Bradford (BioRad). Then, by interpolation with a calibration curve obtained with a standard albumin quantity, the protein concentration of the samples was found. The obtained samples were then subjected to mono-dimensional and Western Blot electrophoretic separation.

The confocal immunofluorescence experiments on the HeLa cells were conducted by using an anti-F-actin antibody, marker of the axonal growth cones (Fan & Simard 2002). The transfection with tag-a-SMN, but not that with the empty vector nor with tag-FL-SMN, causes in the HeLa cells the formation of cellular processes similar to filopodia, relatively long and positive for anti-F-actin marking (FIG. 3h-j). The capacity to induce the formation of immunoreactive filopodia in cells phenotypically lacking neuritic extensions demonstrates the functional importance of a-SMN, since its dominant effect on the formation of structures similar to the growth cones is even exerted on cells lacking this biological characteristic.

The transfection with fusion protein tag-FL-SMN or tag-a-SMN permitted us to distinguish, both in the immunofluorescence experiments and in Western Blot experiments, the levels of endogenous protein from those exogenous, since the anti-tag antibodies specifically recognise a sequence present exclusively in the transfected protein. This alteration of the native protein form, represented by the presence of the tag in amino-terminal position, has inclined us to believe that the biological effect observed following the transfection of tag-a-SMN was due to the presence of the fusion protein and not of the native a-SMN protein. To exclude this possibility, the cDNA of FL-SMN and a-SMN were cloned in the bicistronic vector pIRES-EYFP, which permits separately producing, once transferred in vitro, both the fluorescent protein (EYFP) and the protein of interest. The fluorescent protein is a marker which permits distinguishing the transfected cells from the others, without however interfering with the protein of interest.

The transfected NCS34 cells are intensely fluorescent (in green), due to the presence of the protein EYFP (FIG. 3d). The NDC34 transfected with the native protein FL-SMN, and detected with anti-SMN antibody, does not present changes of the cell morphology and the transfected protein is accumulated at the level of the cytoplasmic granules, which do not seem to have a precise final destiny in the cell. In the cells transfected with a-SMN, the formation is instead observed of new neurites which extend until they come into contact with the cell bodies of the adjacent motor neurons (FIG. 3e-f).

The transfection with the pIRES-EYFP clones was also carried out in HeLa cells. Also in this cell line, the transfected cells show a clear green autofluorescence which permits distinguishing them from the untransfected cells. The HeLa transfected with FL-SMN are characterised by the presence of cytoplasmic granules, immunoreactive to the anti-SMN antibody, inside of which the transfected protein is accumulated. It is moreover possible to observe that the cells transfected with the full length protein do not have morphological alterations. The a-SMN protein is instead capable of modifying the morphology of the HeLa, inducing the growth on the cell membrane of a large number of filopodia.

A similar cellular localisation and an analogous effect on the neuritogenesis was also observed by transfecting the human A-SMN protein (ha-SMN) inserted in an N-terminal GFP vector in NSC34 cells (FIG. 4a-c). The cDNA of the human protein was cloned inside the expression vector GFP-Fusion-TOPO which produces a fluorescent fusion protein resulting from the joining of the GFP protein (green fluorescent protein) with the cloned protein, in the specific case ha-SMN. In this manner, it is possible to visualise the transfected protein (GFP-ha-SMN) without the need of using specific antibodies. The cell protein extracts obtained from NSC34 cells after the transfection were used for Western Blot experiments. Analysing the immunoreactivity of the protein extracts of non-transfected NSC34 cells, it is possible to observe the presence of a single band, with apparent molecular weight of 38 kDa, corresponding to the endogenous FL-SMN protein. The same result is also observed for the protein extracts of NSC34 transfected with the empty vector. Regarding the cell extracts of NSC34 transfected with GFP-ha-SMN, the presence is observed of two immunoreactive bands: the band with greater apparent molecular weight (about 51 kDa, in fact the contribution of the protein GFP us is about 23 kDa) corresponds to the transfected fusion protein GFP-ha-SMN, the band with lower apparent molecular weight (38 kDa) instead corresponds with the endogenous protein FL-SMN (FIG. 4a). Regarding the confocal immunofluorescence experiments, it is possible to observe that the biological effect exerted by ha-SMN in motor neuronal cells NSC34 consists of the induction of the radial growth of long neuritic extensions (200-250 microns), which come into contact with the cell bodies of the adjacent motor neurons. Both on the level of the cell bodies and the neuritic extensions there is an intense fluorescence, due to the presence of the chimeric protein (FIG. 4b-c).

The images of confocal immunofluorescence show that the overexpression of the protein ha-SMN induces the massive growth of extensions similar to neurites on the cell surface. These extensions show an intense immunoreactivity to the anti-ha-SMN antibody and also to the anti-GAP43 antibody, which is a marker of the axonal growth cones. The protein B-50 (Zwiers et al, 1976, 1980), also called GAP-43 (anti growth associated protein 43) (Skene & Willard 1981), is a substrate protein of the PKC (protein kinase C) and is selectively expressed in the growth axons (Van Hooff et al. 1989), at the region near the cell body, while it is not present in the growth cones of the dendrites. The overlapping of the signals related to the immunoreactivity to the anti-ha-SMN and anti-GAP43 antibodies demonstrates that there is an intense colocalisation of the two proteins at the level of the new generation growth cones. This data demonstrates that the extensions present at the cell membrane level of the HeLa cells, following the overexpression of the a-SMN protein, are in fact axonal processes.

It was moreover demonstrated that the biological effect exerted by the protein a-SMN is dominant, since it is capable of inducing the growth of extensions which are immunoreactive to the antibody anti-F-actin, axonal growth marker, also in cells which have no neuronal phenotype (FIG. 4d-f).

To verify the kinetics of the protein expression, we have carried out a transient transfection time course experiment on cells NSC34 for the a-SMN human protein (FIG. 5). After 12-24 hours, the transfected motor neurons have a multipolar aspect with neurites which extend in all directions (FIG. 5a-b). After 48 hours the number of neurons is diminished, the cells are bipolar, but the length of the neurites is considerably increased (FIG. 5c). Finally, after 72 hours the transfected cells have a unipolar aspect, with very long single neurites (FIG. 5d).

On the other hand, by transfecting FL-SMN, no increase is observed of the axon length but rather a reduction after 72 hours of transfection. The statistical analysis of the obtained data shows significant differences in the axonal length (P=2.14×10⁻¹¹) and as a function of the time (P=7.77×10⁻⁹) between the a-SMN and FL-SMN groups. This shows that a-SMN has an effect on the neuritogenesis and that this effect is time-dependent (FIG. 5e). The Western Blot analysis demonstrates that the progressive development of the axonogenesis is associated with the synthesis of the protein a-SMN, which is equally recognised by anti-SMN, anti-tag and human anti-a-SMN antibodies (FIG. 5f).

At this point, in order to identify the epitope responsible for the axonogenesis induction, a functional mapping experiment was carried out of a-SMN by transfecting the motor neurons NSC34 with different constructs containing a N-terminal tag (FIG. 6). The overexpression of the construct containing an in frame stop codon at the first start codon (i.e. which determines the synthesis of the mRNA—see FIG. 6g—but not the related protein) does not induce any morphological change (FIG. 6a).

The overexpression of the peptide encoded by the intron3 determines the accumulation of large cytoplasmic granules without modifications of the cell morphology (FIG. 6b). The peptide encoded by exon 1/2a is accumulated at the level of the neuritic extensions without, however, determining modifications of the axonal growth (FIG. 6c). On the other hand, the overexpression of exon 1/2a/2b or exon 1/2a/2b/3 leads to the accumulation of the protein corresponding to the level of the neurites and to the induction of the axonal growth (FIG. 6d-e).

WB experiments show protein bands by the expected molecular weight deriving from the various constructs of a-SMN (FIG. 6f). Statistical analysis reveals a significant axonogenic effect (p<0.0001) determined by the a-SMN construct with respect to FL-SMN, while the difference between a-SMN, a-SMN exon 1/2a/2b and a-SMN exon 1/2a/2b/3 are not significant (FIG. 6h). The obtained data demonstrates that the synthesis of the a-SMN protein is necessary for the axonal sprouting, that the sequence exon 1-2a is important for the axonal localisation, and that the C-terminal portion is essential for the axonogenesis. The retention of the intron 3 is important only for providing a stop codon necessary for producing an axonogenic polypeptide interrupted at the ex 3/ex 4 junction. This explains the divergence in the amino acid sequence encoded by the intron 3 of man, mouse and rat.

In detail, the transfection was carried out following the Lipofectamina-Plus Reagent (Invitrogen) protocol. The Lipofectima reagent is a liposome formulation capable of interacting with the negative charges of the DNA, previously complexed with Plus Reagent, and to form a lipid-DNA complex which penetrates into the culture cells with high efficiency, subsequently leading them to express the introduced DNA. For the transfection in NSC34 cells, the plasmids pcDNA4 and PIRES-EYFP were used, at whose interior the fragment of interest was previously cloned. In order to carry out the immunocytochemical experiments (ICC) on transfected cells, the cells themselves were placed two days before the transfection in multiwell plates with 6 wells, inside of which, before the placement, a sterilised cover glass is placed on which the cells adhere; the cells were placed at a density of 120,000 cells/well so to obtain a confluence of 70% on the date of transfection. For each well, a DNA quantity was used equal to 5 μg/μl, which was diluted in 65 μl of D-MEM, added with 1% L-Glutamine and lacking antibiotics and serum, and left to incubate for 15 minutes at room temperature after the addition of Plus Reagent (30 μl). Also the Lipofectima (20 μl for every well) was diluted in 80 μl of D-MEM, added with 1% L-Glutamine and lacking antibiotics and serum. The DNA solution, complexed with Plus Reagent, and the diluted Lipofectamina were joined together and incubated for 15 minutes at room temperature. During the incubation, the culture medium in the wells of the multiwell was substituted with 800 μl of D-MEM, added with 1% L-Glutamine and lacking antibiotics and serum. At the end of the incubation, 200 μl of the DNA-Plus-Lipofectamina mixture was added and left in incubation for 3 hours at 37° C. At the end of this time period, the transfection medium was removed and 2 ml of D-MEM added with 1% L-Glutamine and 10% serum was added. After 24-48-72 hours, the cells were fixed in the following manner: the medium was accurately aspirated and the cells were subjected to a quick washing with 1× PBS (0.01 phosphate buffer, 0.0027 KCl, 0.137 NaCl, pH 7.4, preserved at 4° C.) containing Ca²⁺ and Mg²⁺ ions, heated to 37° C.

In every well, 1 ml of 4% paraformaldehyde was added, along with 4% saccharose, and the cells were left to incubate for 25 minutes at room temperature. Once the incubation was terminated, the 4% paraformaldehyde and 4% saccharose solution was aspirated and substituted with 1× PBS lacking Ca²⁺ and Mg²⁺ ions, preserved up until that moment at 4° C. The cells fixed in this manner can be subjected to immunocytochemical experiments.

The cells are incubated with 100% methanol at 20° C. for 10 minutes, then washed three times (10 minutes for each washing) with a low salt concentration buffer (LS: 150 mM NaCl and 10 mM PB at pH 4) and three times (10 minutes for each washing) with a high salt concentration buffer (HS: 500 mM NaCl and 20 mM PB at pH 7.4). To avoid the crosslink with non-specific epitopes, the cells were incubated in goat serum dilution buffer (1× GSDB, 3% normal goat serum, 0.1% Triton X-100, 500 mM NaCl and 20 mM PB, pH 7.4) for 30 minutes. The following primary antibodies were then added: monoclonal TL anti-SMN (diluted 1:1000), monoclonal anti-tag (diluted 1:1000), polyclonal anti-ha-SMN (No. 910, diluted 1:1000), monoclonal anti-F-actin (diluted 1:1000) and monoclonal anti-GAP43 (diluted 1:1000) in 1× GSDB, and the cells were left to incubate overnight. The following day, three washings were carried out (10 minutes for each washing) with HS and subsequently the cells were incubated with the fluorescent secondary antibodies Alexa Fluor® 546 or Alexa Fluor® 488 (Molecular Probes, Eugene, Oreg., USA; diluted 1:2000) for one hour. After three washings with HS (5 minutes for each washing), and three washings with LS (5 minutes for each washing), the cover glasses, containing the cells, were finally mounted with Fluorsave (Calbiochem) and examined with confocal microscope BioRad Radiance 2100. The images were subsequently processed with the Adobe Photoshop 7.0 program. The slides were then preserved at 4° C., in the dark, to minimise the decay of the fluorescent signal.

### BIBLIOGRAPHY

Azzouz M, Ralph G S, Storkebaum E, Walmsley L E, Mitrophanous K A, Kingsman S M, Carmeliet P, Mazarakis N D (2004a) Nature 429:413-417.

Azzouz M, Le T, Ralph G S, Walmsley L, Monani U R, Lee D C, Wilkes F, Mitrophanous K A, Kingsman S M, Burghes A H et al. (2004b) J Clin Invest 114:1726-1731.

Battaglia G, Princivalle A, Forti F, Lizier C, Zeviani M (1997) Hum Mol Genet 6:1961-1971.

Carissimi C, Baccon J, Straccia M, Chiarella P, Maiolica A, Sawyer A, Rappsilber J, Pellizzoni L (2005) FEBS Lett 579:2348-2354.

Carrel T L, McWhorter M L, Workman E, Zhang H, Wolstencroft E C, Lorson C, Bassell G J, Burghes A H, Beattie C E. (2006) J Neurosci 26:11014-11022.

Cashman N R, Durham H D, Blusztajn J K, Oda K, Tabira T, Shaw I T, Dahrouge S, Antel J P (1992) Dev Dyn 194:209-221.

Cifuentes-Diaz C, Nicole S, Velasco M E, Borra-Cebrian C, Panozzo C, Frugier T, Millet G, Roblot N, Joshi V, Melki J (2002) Hum Mol Genet 11:1439-1447.

Cisterni C, Kallenbach S, Jordier F, Bagnis C, Pettmann B (2001) Neurobiol Dis 8:240-251.

Coovert D D, Le T T, McAndrew P E, Strasswimmer J, Crawford T O, Mendell J R, Coulson S E, Androphy E J, Prior T W, Burghes A H M (1997) Hum Mol Genet 6:1205-1214.

Fan L, Simard L R (2002) Hum Mol Genet 11:1605-1614.

Feldkotter M, Schwarzer V, Wirth R, Wienker T F, Wirth B (2002) Am J Hum Genet 70:358-368.

Fischer U, Liu Q, Dreyfuss G (1997) Cell 90:1023-1029.

Gennarelli M, Lucarelli M, Capon F, Pizzuti A, Merlini L, Angelini C, Novelli G, Dallapiccola B (1995) Biochem Biophys Res Commun 213:342-348.

Grimmler M, Otter S, Peter C, Muller F, Chiari A, Fischer U (2005) Hum Mol Genet 14:3099-3111.

Le T T, Pham L T, Butchbach M E, Zhang H L, Monani U R, Coovert D D, Gavrilina T O, Xing L, Bassell G J, Burghes A H (2005) Hum Mol Genet 14:845-857.

Lefebvre S, Burglen L, Reboullet S, Clermont O, Burlet P, Viollet L, Benichou B, Cruaud C, Millasseau P, Zeviani M et al. (1995) Cell 80:155-165.

Lefebvre S, Burlet P, Liu Q, Bertrandy S, Clermont O, Munnich A, Dreyfuss O, Melki J (1997) Nature Genet 16:265-269.

Lefebvre S, Burglen L, Frezal J, Munnich A, Melki J. Hum Mol Genet 1998; 7:1531-6.

Lippa C F, Smith T W (1988) Acta Neuropathol 77:91-94.

Liu Q, Dreyfuss G (1996) EMBO J 15:3555-3565.

Liu Q, Fischer U, Wang F, Dreyfuss G (1997) Cell 90:1013-1021.

Lorson C L, Stasswimmer J, Yao J M, Baleja J D, Hahnen E, Wirth B, Le T, Burghes A H, Androphy E J (1998) Nat Genet 19:63-66.

Lorson C L, Hahnen E, Androphy E J, Wirth B (1999) Proc Natl Acad Sci USA 96:6307-6311.

Lorson C L, Androphy E J. Hum Mol Genet 2000; 9:259-265.

McWhorter M L, Monani U R, Burghes A, Beattie C E (2003) J Cell Biol 162:919-931.

Pagliardini S, Giavazzi A, Setola V, Lizier C, Di Luca M, DeBiasi S, Battaglia G (2000) Hum Mol Genet 9:47-56.

Paushkin S, Gubitz A K, Massenet S, Dreyfuss G (2002) Curr Opin Cell Biol 14:305-312.

Pearn J (1980) Classification of spinal muscular atrophies. Lancet 1:919-922.

Pellizzoni L, Kataoka N, Charroux B, Dreyfuss G (1998) Cell 95:615-624.

Pellizzoni L, Charroux B, Rappsilber J, Mann M, Dreyfuss G (2001) J Cell Biol 152:75-85.

Pisano C, Kollar P, Gianni M, Kalac Y, Giordano V, Ferrara F F, Tancredi R, Devoto A, Rinaldi A, Rambaldi A et al. (2002) Blood 100:3719-3730.

Rossoll W, Kroning A K, Ohndorf U M, Steegborn C, Jablonka S, Sendtner M (2002) Hum Mol Genet 11:93-105.

Rossoll W, Jablonka S, Andreassi C, Kroning A K, Karle K, Monani U R, Sendtner M (2003) J Cell Biol 163:801-812.

Sharma A, Lambrechts A, Hao le T, Le T T, Sewry C A, Ampe C, Burghes A H, Morris G E (2005) Exp Cell Res 309:185-197.

Simeoni S, Mancini M A, Stenoien D L, Marcelli M, Weigel N L, Zanisi M, Martini L, Poletti A (2000) Hum Mol Genet 9:133-144.

Skene J H P, Willard M (1981) J Cell Biol 89:96-103.

Strasswimmer J, Lorson C L, Breiding D E, Chen J J, Le T, Burghes A H, Androphy E J (1999) Hum Mol Genet 8:1219-1226.

Van Hooff C O M, De Graan P N E, Oerstreicher A B, Gispen W H (1989) J Nerosci 8:1789-1795.

Vitali T, Sossi V, Tiziano F, Zappata S, Giuli A, Paravatou-Petsotas M, Neri G, Brahe C (1999) Hum Mol Genet 8, 2525-2532.

Williams B Y, Hamilton S L, Sarkar H K (2000) FEBS Lett 470:207-210.

Yong J, Wan L, Dreyfuss G (2004) Trends Cell Biol 14:226-232.

Young P J, Day P M, Zhou J, Androphy E J, Morris G E, Lorson C L (2002) J Biol Chem 277:2852-2859.

Zhang H L, Pan F, Hong D, Shenoy S M, Singer R H, Bassell G J (2003) J Neurosci 23:6627-6637.

Zhang H, Xing L, Rossoll W, Wichterle H, Singer R H, Bassell G J (2006) J Neurosci 26:8622-8632.

Zwiers H, Velduis M D, Schotman P. Gispen W H (1976) Neurochem Res 1:669-677.

Zwiers H, Schotman P, Gispen W H (1980) J Neurochem 34:1688-1699.

1. Proteins and/or peptides characterised in that they originate from the gene which results from the retention of the intron 3 of the gene SMN identified in the gene bank with the access number AY876898, with use for the diagnosis and/or prevention and/or treatment of neurodegenerative diseases.

2. Proteins and/or peptides according to claim 1, characterised in that they are obtained by means of synthesis or genetic engineering techniques.

3. Proteins and/or peptides according to claim 1, characterised in that they contain one or more amino acid residues in right-handed form.

4. Use of proteins and/or peptides according to claim 1 for the preparation of a drug for the prevention and/or treatment of neurodegenerative diseases.

5. Use of proteins and/or peptides according to claim 1 wherein said neurodegenerative diseases comprise spinal muscular atrophy (SMA) or amyotrophic lateral sclerosis (ALS).

6. Use of proteins and/or peptides according to claim 4 wherein said neurodegenerative diseases comprise the neuronal degeneration which follows trauma.

7. Polyclonal or monoclonal antibodies to proteins and/or peptides in accordance with claim 1, with use for the diagnosis and/or prevention and/or treatment of neurodegenerative diseases.

8. Gene constructs characterised in that they transport proteins and/or peptides in accordance with claim 1 and/or their parts and/or their derivatives.

9. Gene constructs characterised in that they transport further proteins and/or peptides which interact with proteins and/or peptides in accordance with claim 1 and/or with their parts and/or derivatives.

10. Gene constructs according to claim 8 characterised in that they are of human origin.

11. Use of gene constructs according to claim 8 for the preparation of a drug for the prevention and/or treatment of neurodegenerative diseases.

12. Product for the treatment of neurodegenerative diseases which is characterised in that it comprises gene constructs in accordance with claim 8 in association with proteins and/or growth factors which favour its biological activity.

13. Cell lines transfected and/or cotransfected with one or more gene constructs in accordance with claim 8 with use as experimental models for the study of neurodegenerative diseases.

14. Cell lines transfected and/or cotransfected with one or more gene constructs in accordance with claim 8 with use for the preparation of a drug for the prevention and/or treatment of neurodegenerative diseases.

15. Bacterial strains engineered with one or more gene constructs in accordance with claim 8, with use for the production of said proteins and/or peptides.

16. Product for the treatment of neurodegenerative diseases characterised in that it comprises cells for autologous transplant, transfected and/or cotransfected in vitro with one or more gene constructs in accordance with claim 8.

17. Use of gene constructs in accordance with claim 8 for the generation of viral vectors to be used in the gene therapy of neurodegenerative diseases.

18. A screening method for the diagnosis and/or prevention and/or determination of the risk of neurodegenerative diseases carried out on biological material obtained from human organisms, based on the research of proteins and/or peptides originating from the gene which results from the retention of the intron 3 of the gene SMN identified in the gene bank with the access number AY876898.

19. A transgenic, non-human mammal carrier in heterozygotic or homozygotic form of one or more of the gene sequences transported by gene constructs in accordance with claim 8, with use as experimental model for the study of neurodegenerative diseases.

20. A transgenic mammal in accordance with claim 19 characterised in that it is a rodent.

21. A transgenic mammal in accordance with claim 20 characterised in that it is a mouse.

22. A non-human knockout mammal for the intron 3 of the gene SMN identified in the gene bank with the access number AY876898 with use as experimental model for the study of neurodegenerative diseases.

23. A knockout mammal according to claim 22 characterised in that it is a rodent.

24. A knockout mammal according to claim 23 characterised in that it is a mouse.

25. Organs, tissues or cells in vitro deriving from a transgenic animal in accordance with claim 19.

26. Polyclonal or monoclonal antibodies to proteins and/or peptides in accordance with claim 1, with use for the in vitro and in vivo detection of axonal and dendritic extensions.

27. Proteins and/or peptides according to claim 2, characterised in that they are conjugated to peptide sequences with intracellular carrier function, such as but not limited to the peptide TAT, with use for the prevention and treatment of neurodegenerative diseases in humans and in experimental animals.
